SEQUENCE LISTING (14 sequences)

SEQ ID NO: 1 - protein, 8 aa, Artificial Sequence
  Heavy chain complementarity-determining region 1 for antibody or antigen-binding fragment that specifically binds CLDN18.2.
GFSFSSFG

SEQ ID NO: 2 - protein, 8 aa, Artificial Sequence
  Heavy chain complementarity-determining region 2 for antibody or antigen-binding fragment that specifically binds CLDN18.2.
ISSGSRTI

SEQ ID NO: 3 - protein, 11 aa, Artificial Sequence
  Heavy chain complementarity-determining region 3 for antibody or antigen-binding fragment that specifically binds CLDN18.2.
TRYYYGNSFDY

SEQ ID NO: 4 - protein, 12 aa, Artificial Sequence
  Light chain complementarity-determining region 1 for antibody or antigen-binding fragment that specifically binds CLDN18.2.
QSLFNTGNQKNY

SEQ ID NO: 5 - protein, 3 aa, Artificial Sequence
  Light chain complementarity-determining region 2 for antibody or antigen-binding fragment that specifically binds CLDN18.2.
WAS

SEQ ID NO: 6 - protein, 9 aa, Artificial Sequence
  Light chain complementarity-determining region 3 for antibody or antigen-binding fragment that specifically binds CLDN18.2.
QNNYNFPLT

SEQ ID NO: 7 - protein, 118 aa, Artificial Sequence
  Amino acid sequence of the murine heavy chain variable region for an antibody or antigen-binding fragment that specifically binds CLDN18.2.
QVQLKESGGNLVQPGGSRKLSCAASGFSFSSFGMHWVRQAPEKGLEWVAYISSGSRTIYYADTVKGRFTISRDNAKNTLFLQMTSLRSEDTAMYYCTRYYYGNSFDYWGQGTTLTVSS

SEQ ID NO: 8 - protein, 113 aa, Artificial Sequence
  Amino acid sequence of the light chain variable region for a murine antibody or antigen-binding fragment that specifically binds CLDN18.2.
DIVMTQAPSSLTVTTGEKVTLACKSSQSLFNTGNQKNYLTWYQQKPGQPPKLLIYWASTRESGVPDRFTGSGSGTAFTLTISSVQAEDLAVYYCQNNYNFPLTFGAGTKLELK

SEQ ID NO: 9 - protein, 448 aa, Artificial Sequence
  Amino acid sequence of the heavy chain variable region for a chimeric antibody or antigen-binding fragment that specifically binds CLDN18.2.
QVQLKESGGNLVQPGGSRKLSCAASGFSFSSFGMHWVRQAPEKGLEWVAYISSGSRTIYYADTVKGRFTISRDNAKNTLFLQMTSLRSEDTAMYYCTRYYYGNSFDYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, Artificial Sequence
  Amino acid sequence of the light chain variable region for a chimeric antibody or antigen-binding fragment that specifically binds CLDN18.2.
DIVMTQAPSSLTVTTGEKVTLACKSSQSLFNTGNQKNYLTWYQQKPGQPPKLLIYWASTRESGVPDRFTGSGSGTAFTLTISSVQAEDLAVYYCQNNYNFPLTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 118 aa, Artificial Sequence
  Amino acid sequence of the heavy chain variable region for a humanized antibody or antigen-binding fragment that specifically binds CLDN18.2.
QVQLVESGGGLVKPGGSLRLSCAASGFSFSSFGMHWVRQAPGKGLEWVAYISSGSRTIYYADTVKGRFTISRDNAKNSLYLQMNSLRAEDTAVYYCTRYYYGNSFDYWGQGTTVTVSS

SEQ ID NO: 12 - protein, 113 aa, Artificial Sequence
  Amino acid sequence of the light chain variable region for a humanized antibody or antigen-binding fragment that specifically binds CLDN18.2.
DIQMTQSPSSLSASVGDRVTITCKSSQSLFNTGNQKNYLTWYQQKPGKAPKLLIYWASTRESGVPSRFSGSGSGTDFTLTISSLQPEDFATYYCQNNYNFPLTFGQGTRLEIK

SEQ ID NO: 13 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 14 - protein, 261 aa, Homo sapiens; identical to SEQ ID NO: 13

(19) United States
(12) Patent Application Publication
(10) Pub. No.: US 2025/0346665 A1
(43) Pub. Date: Nov. 13, 2025

(54) ANTI-CLDN18.2 ANTIBODY AND APPLICATION THEREOF

(71) Applicant: BEIJING IMMUNOAH PHARMATECH CO., LTD., Beijing (CN)

(72) Inventors: Xiaoyan ZHANG, Beijing (CN); Qinzhi XU, Beijing (CN); Niliang QIAN, Beijing (CN); Cuima YANG, Beijing (CN); Hongjie LI, Beijing (CN); Guili XU, Beijing (CN); Kunlin ZHANG, Beijing (CN); Xin GAO, Beijing (CN)

(21) Appl. No.: 18/263,078

(22) PCT Filed: Jan. 21, 2022
(86) PCT No.: PCT/CN2022/073207

§ 371 Date: Jul. 26, 2023

(30) Foreign Application Priority Data
Jan. 28, 2021 (CN) 202110117724.4

(51) Int. Cl. C07K 16/28; A61P 35/00; C12N 15/63

(52) U.S. Cl. CPC C07K 16/28; A61P 35/00; C12N 15/63; C07K 2317/14; C07K 2317/24; C07K 2317/565

(57) ABSTRACT

Provided are an anti-claudin 18.2 (CLDN18.2) antibody and use thereof. Also provided are a chimeric antibody and a humanized antibody of the anti-CLDN18.2 antibody. These antibodies have high affinity for human CLDN18.2 proteins, and are promising antibody molecules for preventing and/or treating CLDN18.2 related diseases, such as CLDN18.2-related tumors.

### RELATED APPLICATIONS

This application is a 35 U.S.C. § 371 filing of International Application No. PCT/CN2022/073207, filed Jan. 21, 2022, which application claims priority to Chinese Patent Application No. 202110117724.4, filed Jan. 28, 2021. The entire contents of these applications are herein incorporated by reference in their entireties.

### TECHNICAL FIELD

The present application relates to the field of antibodies, and more particularly, to anti-CLDN18.2 antibodies and use thereof.

### SEQUENCE LISTING

The present application is being filed along with a Sequence Listing in computer readable format. The Sequence Listing is provided as a file entitled 744967-INV-431US-SEQUENCE-LISTING REPLACEMENT 03_20_2024.txt created Mar. 20, 2024 and is approximately 17,857 bytes in size. The information in the computer readable format of the sequence listing is incorporated herein by reference in its entirety.

### BACKGROUND OF INVENTION

Cancer has biological characteristics such as abnormal cell differentiation and proliferation, uncontrollable growth, infiltration and metastasis, and its occurrence involves a complex process with multiple factors and multiple steps. The treatment means for cancer includes surgeries, radiotherapies, chemotherapies, immunotherapies, etc. For many patients with intermediate and advanced cancer, immunotherapy greatly improves their survival and quality of life, and the antibody therapy therein has become a recognized immunotherapy that is effective against cancer.

There are about one million new cases of gastric cancer worldwide every year. Although many improvements have been made in conventional cancer treatment methods, the recurrence rate continues to increase, and recurrent patients are susceptible to tolerance to conventional therapies leading to treatment failure. The 5-year survival rate in patients with advanced gastric cancer is approximately 5%-20% and the median overall survival rate is approximately 10 months. The 5-year overall survival rate of pancreatic cancer is approximately 6˜8%. The poor prognosis of both cancers has prompted researchers to look for new therapeutic targets.

In 1998, Shorichiro Tsukita et al. first discovered claudin (CLDN), a major transmembrane protein component for the tight junction of human endothelium and epithelium, which has the function of maintaining tissue osmotic balance and cell polarity. Tissue-specific expression of CLDN members showed that this protein family not only has an important role in maintaining cell permeability and controlling tight junctions, but is also critical in protein-protein interactions and signaling of tissue-cell contact. There are 27 mammal CLDN genes currently, including three distant members which were discovered recently, but divergence remains as to whether all the three distant members should be classified as CLDN. Not all of these genes are present in all mammals. For example, CLDN13 is present in rodents but not in humans. Thus, a total of 26 CLDN genes have been found in humans up to now.

CLDN18 is one of the few members of the human claudin family that are strictly restricted to one cell line. The CLDN 18 molecule is a four-time transmembrane protein with four transmembrane hydrophobic regions, two of which are extracellular loops, and one intracellular loop with its ends located intracellularly. The molecule has two cleavage isomers, CLDN18.1 and CLDN18.2, resulting from the selective splicing of the first exon, and they are highly homologous to each other in sequence, with only eight amino acid differences. CLDN18.1 is mainly expressed in lung tissue cells; and the expression of CLDN18.2 in normal tissues is strictly limited to differentiated epithelial cells of the gastric mucosa, but not in the gastric stem cell region. CLDN18.2 is retained in malignant transformation and is expressed in a considerable proportion of primary gastric cancers and their metastatic tumors. In addition, CLDN18.2 is ectopically activated in pancreatic, esophageal, ovarian and lung tumors frequently. Clinical data showed that the CLDN18.2-specific human-mouse chimeric monoclonal antibody IMAB362 is not significantly toxic to normal tissues. The overall objective response rate of IMAB362 to patients with gastric cancer was approximately 10%, and these data suggested that CLDN18.2 may be a promising therapeutic target for the treatment of gastric cancer and other CLDN18.2-positive tumors.

### SUMMARY OF INVENTION

To solve the above technical problem, the present application provides an anti-CLDN18.2 antibody and application thereof. Specifically, the present application provides the following technical solutions.

In a first aspect, the present application provides an antibody or an antigen-binding portion thereof that specifically binds to CLDN18.2, comprising a heavy chain variable region comprising any one or more of HCDR1, HCDR2, and HCDR3 sequences, wherein the HCDR1 sequence is GFSFSSFG (SEQ ID NO:1), the HCDR2 sequence is ISSGSRTI (SEQ ID NO:2), and the HCDR3 sequence is TRYYYGNSFDY (SEQ ID NO:3).

In some embodiments, the antibody or an antigen-binding portion thereof further comprises a light chain variable region comprising any one or more of the LCDR1, LCDR2, and LCDR3 sequences, wherein the LCDR1 sequence is QSLFNTGNQKNY (SEQ ID NO: 4), the LCDR2 sequence is WAS (SEQ ID NO: 5), and the LCDR3 sequence is QNNYNFPLT (SEQ ID NO: 6).

In some embodiments, the antibody is a murine antibody. Preferably, the amino acid sequence of the heavy chain variable region is shown in SEQ ID NO:7, and/or the amino acid sequence of the light chain variable region is shown in SEQ ID NO:8.

In some embodiments, the antibody is a chimeric antibody. Preferably, the chimeric antibody comprises a heavy chain having an amino acid sequence as shown in SEQ ID NO:9, and/or a light chain having an amino acid sequence as shown in SEQ ID NO:10.

In some embodiments, the antibody is a humanized antibody. Preferably, the amino acid sequence of the heavy chain variable region is shown in SEQ ID NO:11, and/or the amino acid sequence of the light chain variable region is shown in SEQ ID NO:12.

In a second aspect, the present application provides a pharmaceutical composition comprising the antibody or an antigen-binding portion thereof of the first aspect and a pharmaceutically acceptable carrier.

In a third aspect, the present application provides a nucleic acid molecule encoding the antibody or an antigen-binding portion thereof of the first aspect.

In a fourth aspect, the application provides an expression vector comprising the nucleic acid molecule of the third aspect.

In a fifth aspect, the present application provides a host cell comprising the nucleic acid molecule of the third aspect or the expression vector of the fourth aspect.

In a sixth aspect, the present application provides use of the antibody or an antigen-binding portion thereof of the first aspect, the pharmaceutical composition of the second aspect, the nucleic acid molecule of the third aspect, the expression vector of the fourth aspect, or the host cell of the fifth aspect in the manufacture of a medicament for the prevention and/or treatment of a CLDN18.2-related disease.

In a seventh aspect, the present application provides a method of preventing and/or treating a CLDN18.2-related disease, comprising administering to a subject in need thereof a therapeutically effective amount of the antibody or an antigen-binding portion thereof of the first aspect, the pharmaceutical composition of the second aspect, the nucleic acid molecule of the third aspect, the expression vector of the fourth aspect, or the host cell of the fifth aspect.

In an eighth aspect, the present application provides the antibody or an antigen-binding portion thereof of the first aspect, the pharmaceutical composition of the second aspect, the nucleic acid molecule of the third aspect, the expression vector of the fourth aspect, or the host cell of the fifth aspect for use in preventing and/or treating a CLDN18.2-related disease.

In any one of the sixth to eighth embodiments, the CLDN18.2-related disease is a tumor.

In some embodiments, the tumor is selected from one or more of the following: gastric cancer, pancreatic cancer, intestinal cancer, esophageal cancer, liver cancer, ovarian cancer, lung cancer, bladder cancer, and the like, and metastatic tumors thereof.

In a ninth aspect, the present application provides a detection reagent or a kit comprising the antibody or an antigen-binding portion thereof of the first aspect.

The antibody or an antigen-binding portion thereof that specifically binds to CLDN18.2 of the present application is capable of inducing CLDN18.2 mediated anti-tumor immune response, and/or inhibiting tumor growth, and the like.

### BRIEF DESCRIPTION OF DRAWINGS

FIG. 1 shows the binding affinity of the murine anti-CLDN18.2 antibody (an antibody secreted by the hybridoma 7D10-1) of the present application to HEK293 cells 293T-18.2 overexpressing human CLDN18.2.

FIG. 2 shows the binding affinity of the chimeric anti-CLDN18.2 antibody of the present application to HEK293 cells 293T-18.2 overexpressing human CLDN18.2.

FIG. 3 shows the binding affinity of the humanized anti-CLDN18.2 antibody of the present application to HEK293 cells 293T-18.2 overexpressing human CLDN18.2.

FIG. 4 shows the binding affinity of the humanized anti-CLDN18.2 antibody of the present application to the human gastric cancer cell line NUGC4.

FIG. 5 shows the ADCC activity of the humanized anti-CLDN18.2 antibody of the present application.

### DETAILED DESCRIPTION OF INVENTION

The following definitions and methods are provided to better define the application and to guide those of ordinary skill in the art in the practice of the application. Unless otherwise indicated, the terms of the application are to be understood in accordance with the usual usage of one of ordinary skill in the relevant art.

### Definitions

As used herein, the term “antibody” refers to an immunoglobulin molecule comprising four polypeptide chains, i.e., two heavy chains (H) connected to each other via disulfide bonds and two light chains (L), and multimers (e.g., IgM) thereof. Each heavy chain contains a heavy chain variable region (abbreviated as VH) and a heavy chain constant region (abbreviated as CH). The heavy chain constant region contains three domains, CH1, CH2 and CH3. Each light chain contains a light chain variable region (abbreviated as VL) and a light chain constant region (abbreviated as CL). The light chain constant region contains one domain (CL1). The VH and VL regions can be further subdivided into hypervariable regions referred to as complementarity determining regions (CDRs) in which conserved regions referred to as framework regions (FRs) are inserted therein. In some embodiments, the light and heavy chain variable domains each comprise FR1, CDR1, FR2, CDR2, FR3, CDR3, FR4 from the N-terminus to the C-terminus.

As used herein, the term “antigen-binding portion” of an antibody refers to a portion or a segment of an intact antibody molecule responsible for binding to an antigen. The antigen-binding portion may comprise a heavy chain variable region (VH), a light chain variable region (VL), or both. The antigen-binding portion of an antibody may be prepared from the intact antibody molecule using any suitable standard technique, including proteolytic digestion or recombinant genetic engineering techniques, and the like. Non-limiting examples of the antigen-binding portion include Fab fragments, F(ab′)₂ fragments, Fd fragments, Fv fragments, single chain Fv (scFv) molecules, single domain antibodies, dAb fragments, and minimal recognition units (e.g., isolated CDRs) consisting of amino acid residues that mimic the hypervariable region of an antibody. The term “antigen-binding portion” also includes other engineered molecules such as diabodies, triantibodies, tetra-antibodies, and micro-antibodies, among others. For example, the Fd fragment herein refers to an antibody fragment consisting of the VH and CH1 domains; The Fv fragment consists of the VL and VH domains in a single arm of an antibody; The dAb fragment (Ward et al., Nature 1989; 341:544-546) consists of the VH domain.

It is well known to those skilled in the art that the complementarity determining regions (CDRs, typically CDR1, CDR2 and CDR3) are the regions of the variable region that have the greatest impact on the affinity and specificity of an antibody. The CDR sequences of VH or VL are commonly defined in two ways, namely the Kabat definition and the Chothia definition, see, for example, Kabat et al., “Sequences of Proteins of Immunological Interest”, National Institutes of Health, Bethesda, MD. (1991); Al-Lazikani et al., J Mol Biol 273:927-948 (1997); and Martin et al., Proc. Natl. Acad. Sci. USA 86:9268-9272 (1989). For a given antibody variable region sequence, the CDR region sequences in the VH and VL sequences may be determined according to the Kabat definition or the Chothia definition. In an embodiment of the present application, a CDR sequence is defined using Kabat. Herein, CDR1, CDR2 and CDR3 of the heavy chain variable region are simply referred to as HCDR1, HCDR2 and HCDR3, respectively; and CDR1, CDR2, and CDR3 of the light chain variable region are simply referred to as LCDR1, LCDR2, and LCDR3, respectively.

For the variable region sequence of a given antibody, the CDR region sequences in the variable regions may be analyzed in a number of ways, for example, may be determined using the online software Abysis (http://www.abysis.org/).

As used herein, the term “specifically bind to” refers to a non-random binding reaction between two molecules, e.g., the binding of an antibody to an epitope, e.g., the ability of an antibody to bind to a specific antigen with an affinity that is at least two times greater than its affinity for a non-specific antigen. However, it will be appreciated that antibodies are capable of specifically binding to two or more antigens associated with their sequences. For example, the antibodies of the present application may specifically bind to human CLDN18.2.

As used herein, the term “monoclonal antibody” refers to an antibody obtained from a population of substantially homogeneous antibodies, i.e., each antibody in the population is the same except that naturally occurring mutations may be present in a small number of antibodies. Monoclonal antibodies herein include, in particular, “chimeric” antibodies in which a portion of the heavy and/or light chains are identical or homologous to corresponding sequences in antibodies derived from a particular species or belonging to a particular antibody class or subclass, while the remainder of the heavy and/or light chains are identical or homologous to corresponding sequences in antibodies derived from another species or belonging to another antibody class or subclass, and also include fragments of such antibodies as long as they exhibit the desired biological activity (see, U.S. Pat. No. 4,816,567; and Morrison et al., Proc. Natl. Acad. Sci. USA 81:6851-6855 (1984)).

As used herein, the term “murine antibody” refers to any antibody in which all domain sequences are mouse sequences. Such antibodies may be produced by hybridomas.

As used herein, the term “chimeric antibody” refers to an antibody comprising a segment from two or more different antibodies. In some embodiments, one or more CDRs are derived from a mouse anti-CLDN18.2 antibody. In other embodiments, all CDRs are derived from a mouse anti-CLDN18.2 antibody. In some embodiments, CDRs from more than one mouse anti-CLDN18.2 antibody are combined in a chimeric antibody. For example, the chimeric antibody may comprise CDR1 in the light chain from a first mouse anti-CLDN18.2 antibody, CDR2 in the light chain from a second mouse anti-CLDN18.2 antibody, and CDR3 in the light chain from a third mouse anti-CLDN18.2 antibody, and the CDRs from the heavy chain may be derived from one or more other anti-CLDN18.2 antibodies. In addition, the framework region may be derived from the same anti-CLDN18.2 antibody or from one or more different individuals. In some embodiments, a chimeric antibody herein comprises a variable region of a murine antibody (including a heavy chain variable region VH and/or a light chain variable region VL) and a constant region of a human antibody.

As used herein, the term “humanized antibody” refers to a CDR-grafted antibody, specifically to an antibody produced by grafting a murine CDR region sequences into the framework of a human antibody variable region. The aim is to overcome the fact that chimeric antibodies induce strong immune side effects in humans by carrying a large number of protein components of other species, such as mice.

As used herein, the term “nucleic acid molecule” may refer to both a DNA molecule and an RNA molecule, either single-stranded or double-stranded. The nucleic acid molecule may also be cDNA.

As used herein, the term “CLDN18.2-related diseases” includes diseases and/or conditions associated with CLDN18.2 signaling pathways. Exemplary CLDN18.2-related diseases or conditions include tumors, cancer such as gastric cancer, pancreatic cancer, colon cancer, esophageal cancer, liver cancer, ovarian cancer, lung cancer, and bladder cancer, as well as metastatic cancer described above.

As used herein, the term “EC50” refers to the concentration at which 50% of the maximum effect is induced, i.e., the concentration for 50% of maximal effect, EC50.

As used herein, the term “immune response” refers to a biological response within a vertebrate against an extraneous agent that protects the organism against such agents and diseases caused thereby. The immune response, mediated by the action of cells of the immune system (e.g., T lymphocytes, B lymphocytes, natural killer (NK) cells, macrophages, eosinophils, mast cells, dendritic cells, or neutrophils) and any of such cells or soluble macromolecules produced by the liver (including antibodies, cytokines, and complements), results in selective targeting, binding, damage, destruction, and/or elimination of invaded pathogens, pathogen-infected cells or tissues, cancerous or other abnormal cells from vertebrates, or normal human cells or tissues in the context of autoimmune or pathological inflammation. Immune responses include activation or inhibition of T cells (e.g., effector T cells) or Th cells (e.g., CD4⁺ or CD8⁺ T cells) or inhibition of Treg cells.

As used herein, the term “cancer” refers to a broad class of diseases characterized by the uncontrolled growth of abnormal cells in vivo. Dysregulated cell division can result in malignancies or cells that invade adjacent tissues and can metastasize to distant parts of the body via the lymphatic system or blood flow.

As used herein, the term “treatment” refers to any type of interventions or methods practiced on a subject or administering an active agent to a subject, in which the purpose is to reverse, relieve, ameliorate, inhibit, or alleviate or prevent symptoms, complications, conditions, or disease-related progression, development, severity, or recurrence.

As used herein, the term “prevention” refers to administering to a subject not suffering from a disease to prevent the disease or to minimize its effect, if present.

### DETAILED DESCRIPTION

The human CLDN18.2 protein is highly homologous to the human CLDN18.1, and it is a four transmembrane protein with a very short extracellular domain peptide sequence, very low immunogenicity, and very little possibility of producing specific antibodies, so that the probability of screening hybridomas capable of secreting antibodies recognizing CLDN18.2 is very low. A large proportion of the antibodies in the screened hybridoma supernatant are those that bind to both the human CLDN18.2 and the human CLDN18.1. The present inventors have screened a hybridoma cell in which the antibody in the supernatant binds to CLDN18.2-expressing cells and hardly binds to CLDN18.1-expressing cells. Accordingly, the present application provides a novel anti-CLDN18.2 antibody or an antigen-binding portion thereof that specifically binds to CLDN18.2.

The present inventors have also prepared chimeric and humanized antibody forms from the murine anti-CLDN18.2 antibody by genetic engineering means. These antibodies are capable of specifically binding to CLDN18.2 expressed on the cell surface, thereby effectively inducing CLDN18.2-mediated immune responses and preventing or treating CLDN18.2-related diseases.

In a first aspect, the present application provides an antibody or an antigen-binding portion thereof that specifically binds to CLDN18.2, comprising a heavy chain variable region comprising any one or more of HCDR1, HCDR2, and HCDR3 sequences, wherein the HCDR1 sequence is GFSFSSFG (SEQ ID NO:1), the HCDR2 sequence is ISSGSRTI (SEQ ID NO:2), and the HCDR3 sequence is TRYYYGNSFDY (SEQ ID NO:3).

In a preferred embodiment, the heavy chain variable region of the antibody or an antigen-binding portion thereof herein comprises the HCDR1 sequence as shown in SEQ ID NO:1, the HCDR2 sequence as shown in SEQ ID NO:2, and the HCDR3 sequence as shown in SEQ ID NO:3.

In some embodiments, the antibody or an antigen-binding portion thereof herein further comprises a light chain variable region comprising any one or more of LCDR1, LCDR2, and LCDR3 sequences, wherein the LCDR1 sequence is QSLFNTGNQKNY (SEQ ID NO: 4), the LCDR2 sequence is WAS (SEQ ID NO: 5), and the LCDR3 sequence is QNNYNFPLT (SEQ ID NO: 6).

In a preferred embodiment, the light chain variable region of the antibody or an antigen-binding portion thereof herein comprises LCDR1 as shown in SEQ ID NO:4, LCDR2 as shown in SEQ ID NO:5, and LCDR3 as shown in SEQ ID NO:6.

In a preferred embodiment, the antibody or an antigen-binding portion thereof herein comprises a heavy chain variable region and a light chain variable region, wherein the heavy chain variable region comprises HCDR1 as shown in SEQ ID NO:1, HCDR2 as shown in SEQ ID NO:2, and HCDR3 as shown in SEQ ID NO:3, and the light chain variable region comprises LCDR1 as shown in SEQ ID NO:4, LCDR2 as shown in SEQ ID NO:5, and LCDR3 as shown in SEQ ID NO:6.

In some more specific embodiments, the antibodies disclosed herein may be anti-human CLDN18.2 monoclonal antibodies. Types and subtypes of an anti-CLDN18.2 antibody can be determined by any means known in the art. Generally, types and subtypes of an antibody can be determined using antibodies specific for a particular type and subtype of an antibody. The isotype of an anti-CLDN18.2 antibody can be determined using an ELISA assay. For example, human Ig can be identified using murine Ig-adsorbed anti-human lg.

In some embodiments, the antibody herein is a murine antibody. Preferably, the amino acid sequence of the heavy chain variable region is as shown in SEQ ID NO:7, and/or the amino acid sequence of the light chain variable region is as shown in SEQ ID NO:8.

In a preferred embodiment, the murine antibody herein comprises a heavy chain variable region as shown in SEQ ID NO:7 and a light chain variable region as shown in SEQ ID NO:8.

In some embodiments, the antibody herein is a chimeric antibody. The chimeric antibody herein comprises the variable regions of a murine antibody (including a heavy chain variable region VH and/or a light chain variable region VL) and the constant region of a human antibody.

In a preferred embodiment, the chimeric antibody herein comprises the variable regions of a murine antibody (including a heavy chain variable region and a light chain variable region) and the constant region of a human antibody.

Preferably, the chimeric antibody comprises a heavy chain as shown in SEQ ID NO:9, and/or a light chain as shown in SEQ ID NO:10.

In a preferred embodiment, the chimeric antibody herein comprises a heavy chain as shown in SEQ ID NO:9 and a light chain as shown in SEQ ID NO:10.

In some embodiments, the antibody herein is a humanized antibody. The humanized antibody herein comprises the CDR regions of a murine antibody (including any one or more of HCDR1, HCDR2, and HCDR3, and/or any one or more of LCDR1, LCDR2, and LCDR3), the framework regions of a human antibody variable region (including any one or more of FR1, FR2, FR3, and FR4), and optionally the constant region of a human antibody.

In a preferred embodiment, the humanized antibody herein comprises the CDR regions of a murine antibody (including HCDR1, HCDR2, HCDR3, LCDR1, LCDR2, and LCDR3), the framework regions of human antibody variable regions (including FR1, FR2, FR3, and FR4), and optionally the constant region of a human antibody.

Preferably, the amino acid sequence of the heavy chain variable region is shown in SEQ ID NO:11, and/or the amino acid sequence of the light chain variable region is shown in SEQ ID NO:12.

In a preferred embodiment, the humanized antibody herein comprises a heavy chain variable region as shown in SEQ ID NO:11 and a light chain variable region as shown in SEQ ID NO:12.

The antibody herein may also comprise the constant regions of a murine or human antibody. The constant regions of a murine antibody include a heavy chain constant region of murine IgG1, IgG2a, IgG2b, or IgG3, and a light chain constant region of kappa or lambda type. The human antibody constant region includes a heavy chain constant region of human IgG1,IgG2, IgG3, or IgG4, and a light chain constant region of kappa or lambda type.

In some embodiments, the CLDN18.2 herein is a primate CLDN18.2. Preferably, the primate CLDN18.2 herein is selected from human CLDN18.2 or monkey CLDN18.2.In some embodiments, the antigen-binding portion herein is selected from a Fab fragment, a Fab′ fragment, an F(ab′)₂ fragment, an Fv fragment, an scFv fragment, an Fd fragment, or a single domain antibody.

As used herein, the term “Fab fragment” encompasses a light chain and the CH1 and variable regions of a heavy chain. The heavy chain of the Fab molecule cannot form a disulfide bond with another heavy chain molecule.

As used herein, the term “Fab′ fragment” contains a light chain and a portion or a fragment of a heavy chain that contains a VH domain and a CH1 domain and a region between the CH1 and CH2 domains such that an interchain disulfide bond can be formed between two heavy chains of two Fab′ fragments to form an F(ab′)2 molecule.

As used herein, the term “F(ab′)2 fragment” contains two light chains and two heavy chains containing a portion of the constant region between the CH1 and CH2 domains such that an interchain disulfide bond is formed between the two heavy chains. The F(ab′)2 fragment thus consists of two Fab′ fragments which are linked together by a disulfide bond between the two heavy chains.

As used herein, the term “Fv fragment” encompasses variable regions from heavy and light chains, but lacks constant regions.

As used herein, the term “single chain Fv” or “scFv” refers to an antibody fragment comprising the VH and VL domains of an antibody, wherein these domains are present in a single polypeptide chain form. Typically, the Fv polypeptide further comprises a polypeptide linker between the VH and VL domains that enables the scFv to form the desired structure for antigen binding.

As used herein, the term “single domain antibody” refers to an antigen-binding portion comprising a heavy chain variable region (VHH) and two conventional CH2 and CH3 regions. It is a natural antibody without a light chain initially found in the peripheral blood of alpaca. Although the antibody contains only one heavy chain variable region (VHH) and two conventional CH2 and CH3 regions, it does not adhere to each other as easily as an artificially engineered single chain antibody fragment (scFv), or even aggregate into blocks. More importantly, the separately cloned and expressed VHH structure has structural stability and binding activity to the antigen comparable to those of the original heavy chain antibody, and is a known minimum unit capable of binding to a target antigen. The VHH crystal only has the molecular weight of 15 KDa and is therefore also referred to as Nanobody (Nb).

In a second aspect, the present application provides a pharmaceutical composition comprising the antibody or an antigen-binding portion thereof of the first aspect and a pharmaceutically acceptable carrier.

Pharmaceutically acceptable carriers and diluents include saline, aqueous buffer solutions, solvents, and/or dispersion media. The use of such carriers and diluents is well known in the art. Some non-limiting examples of materials useful as pharmaceutically acceptable carriers include: (1) sugars, such as lactose, glucose, and sucrose; (2) starches such as corn starch and potato starch; (3) cellulose and derivatives thereof, such as sodium carboxymethylcellulose, methylcellulose, ethylcellulose, microcrystalline cellulose and cellulose acetate; (4) powdered tragacanth; (5) malt; (6) gelatin; (7) lubricants such as magnesium stearate, sodium lauryl sulfate and talc; (8) excipients such as cocoa butter and suppository waxes; (9) oils such as peanut oil, cottonseed oil, safflower oil, sesame oil, olive oil, corn oil and soybean oil; (10) glycols, such as propylene glycol; (11) polyols such as glycerol, sorbitol, mannitol and polyethylene glycol (PEG); (12) esters such as ethyl oleate and ethyl laurate; (13) agar; (14) buffers such as magnesium hydroxide and aluminum hydroxide; (15) alginic acid; (16) pyrogen-free water; (17) isotonic saline; (18) Ringer's solution; (19) ethyl alcohol; (20) pH buffer solutions; (21) polyesters, polycarbonates and/or polyanhydrides; (22) fillers such as polypeptides and amino acids; (23) serum components such as serum albumin, HDL and LDL; (22) C₂-C₁₂ alcohols such as ethanol; and (23) other non-toxic compatible substances used in pharmaceutical formulations. Wetting agents, colorants, mold release agents, coating agents, sweetening agents, flavoring agents, perfuming agents, preservatives, and antioxidants may also be present in pharmaceutical formulations.

In some embodiments, the pharmaceutical composition herein further comprises one or more other active ingredients, e.g., agents for the treatment of CLDN18.2-related diseases such as tumors.

In a third aspect, the present application provides a nucleic acid molecule encoding the antibody or an antigen-binding portion thereof of the first aspect.

In preferred embodiments, the nucleic acids herein may be codon optimized nucleic acids suitable for expression in host cells. For example, according to the degeneracy of the codon, it still encodes the same protein. Methods for codon optimization according to the host cells used are well known to those skilled in the art.

In a fourth aspect, the present application provides an expression vector comprising the nucleic acid molecule of the third aspect.

Any suitable expression vectors can be used. For example, prokaryotic cloning vectors include plasmids from E. coli, such as colEI, pCRI, pBR322, pMB9, pUC, pKSM, and RP4. Prokaryotic vectors also include phage DNA such as M13 and derivatives of other filamentous single-stranded DNA phages. An example of a vector useful for yeast is the 2μ plasmid. Suitable vectors for expression in mammalian cells include the following well-known derivatives: SV-40, adenovirus, retrovirus-derived DNA sequences, and shuttle vectors derived from combinations of functional mammalian vectors, such as those described above, and functional plasmid and phage DNA.

Additional eukaryotic expression vectors are known in the art (e.g., P J. Southern & P. Berg, J. Mol.Appl. Genet, 1:327-341 (1982); Subramani et al., Mol.Cell. Biol, 1:854-864 (1981); Kaufinann & Sharp, “Amplification And Expression of Sequences Cotransfected with a Modular Dihydrofolate Reductase Complementary DNA Gene,” J. Mol. Biol, 159:601-621 (1982); Kaufhiann & Sharp, Mol. Cell. Biol, 159:601-664 (1982); Scahill et al., “Expression And Characterization Of The Product Of A Human Immune Interferon DNA Gene In Chinese Hamster Ovary Cells,” Proc. Nat'l Acad. Sci USA, 80:4654-4659 (1983); Urlaub & Chasin, Proc. Nat'l Acad. Sci USA, 77:4216-4220, (1980), which is incorporated herein by reference in its entirety).

Expression vectors useful in the application comprise at least one expression control sequence operably linked to a DNA sequence or fragment to be expressed. The control sequence is inserted into a vector to control and regulate the expression of cloned DNA sequences. Examples of useful expression control sequences are the lac system, the trp system, the tac system, the trc system, the major operon and promoter region of the phage Lamda, the control region of the fd coat protein, the glycolytic promoter of the yeast, such as the promoter of 3-phosphoglycerate kinase, the promoter of the yeast acid phosphatase, such as Pho5, the promoter of the yeast alpha mating factor, and promoters derived from a polyomavirus, an adenovirus, a retrovirus, and a simian virus, such as the early and late promoters of SV40, and other sequences known to control gene expression of prokaryotic or eukaryotic cells and viruses or combinations thereof.

In a fifth aspect, the present application provides a host cell comprising the nucleic acid molecule of the third aspect or the expression vector of the fourth aspect.

In some embodiments, the host cells herein are mammal cells. Mammal cells may include, but are not limited to, CHO cells, NSO cells, SP2/0 cells, HEK293 cells, COS cells, and PER.C6 cells. One skilled in the art will be able to select suitable host cells as desired.

A method of preparing an anti-CLDN18.2 monoclonal antibody disclosed herein may comprise culturing the host cells under expression conditions to express the anti-CLDN18.2 monoclonal antibody; and isolating and purifying the expressed anti-CLDN18.2 monoclonal antibody. Using the above method, a crude anti-CLDN18.2 monoclonal antibody can be obtained. The anti-CLDN18.2 monoclonal antibody is then purified to a substantially homogeneous substance by purification methods, including affinity purification based on CLDN18.2, non-denaturing gel purification, HPLC or RP-HPLC, molecular exclusion, purification on a protein A column, or any combination of these techniques, e.g., a single band on SDS-PAGE electrophoresis.

In a sixth aspect, the present application provides use of the antibody or an antigen-binding portion thereof of the first aspect, the pharmaceutical composition of the second aspect, the nucleic acid molecule of the third aspect, the expression vector of the fourth aspect, or the host cell of the fifth aspect in the manufacture of a medicament for the prevention and/or treatment of a CLDN18.2-related disease.

In a seventh aspect, the present application provides a method of preventing and/or treating a CLDN18.2-related disease, comprising administering to a subject in need thereof a therapeutically effective amount of the antibody or an antigen-binding portion thereof of the first aspect, the pharmaceutical composition of the second aspect, the nucleic acid molecule of the third aspect, the expression vector of the fourth aspect, or the host cell of the fifth aspect.

In some embodiments, the method further comprises administering a second agent that prevents and/or treats CLDN18.2-related diseases such as tumors.

As used herein, the term “individual” refers to mammal, including but not limited to primates, cattle, horses, pigs, sheep, goats, dogs, cats, and rodents such as rats and mice. Preferably, mammal is a non-human primate or human. Particularly preferred mammal is human. As used herein, “individual” and “subject” may be used interchangeably.

“Treatment” refers to both therapeutic treatment and prophylactic or preventive measures aimed at preventing or slowing (alleviating) the target pathology state or condition. Subjects in need of treatment include those in which the condition already exists, as well as those in which the condition will develop or is intended to be prevented. Thus, a subject to be treated herein has been diagnosed with or tend to have or predisposed to the condition.

As used herein, a “therapeutically effective amount” can be determined as desired, and one of ordinary skill in the art can readily grasp the amount actually required, for example, depending on the weight, age, and condition of the patient.

In an eighth aspect, the present application provides the antibody or an antigen-binding portion thereof of the first aspect, the pharmaceutical composition of the second aspect, the nucleic acid molecule of the third aspect, the expression vector of the fourth aspect, or the host cell of the fifth aspect for use in preventing and/or treating a CLDN18.2-related disease.

In any one embodiment of the sixth to eighth aspects, the CLDN18.2-related disease is a tumor.

In some embodiments, the tumor herein is primary or metastatic cancers. In particular embodiments, the tumor is selected from lung cancer such as non-small cell lung cancer, colorectal cancer, bladder cancer, hematopoietic cancers such as leukemia, breast cancer, gastric cancer, esophageal cancer, B-lymphocyte non-Hodgkin's lymphoma, Hodgkin's lymphoma, anaplastic large cell lymphoma, head and neck cancer such as head and neck squamous cell carcinoma, malignant glioma, renal cancer, melanoma, prostate cancer, bone cancer, bone giant cell tumor, pancreatic cancer, ovarian cancer, sarcoma, liver cancer, skin squamous cell carcinoma, thyroid cancer, cervical cancer, nasopharyngeal cancer, endometrial cancer, or metastatic cancers of the above tumor.

In embodiments of any aspect, the method, use, and pharmaceutical composition herein may further comprise administering to the subject a second agent and/or treatment, e.g., as part of a combination therapy. Non-limiting examples of the second agent and/or treatment may include radiation therapy, surgery, gemcitabine, cilastatin, paclitaxel, carboplatin, bortezomib, AMG479, vorinostat, rituximab, temozolomide, rapamycin, chlorambucil; gemcitabine; 6-thioguanine; mercaptopurine; methotrexate; platinum analogs such as cisplatin, oxaliplatin and carboplatin; vinblastine; vindesine; Etoposide (VP-16); ifosfamide; mitoxantrone; vincristine; vinorelbine; novantrone; teniposide; edatrexate; daunomycin; aminopterin; xeloda; inhibitors of PKC-α, Raf, H-Ras, EGFR (e.g., Tarceva®), and VEGF-A that reduce cell proliferation, as well as pharmaceutically acceptable salts, acids, or derivatives of any of the above.

In a ninth aspect, the present application provides a detection reagent or a kit comprising the antibody or an antigen-binding portion thereof of the first aspect.

The antibody or an antigen-binding portion thereof herein may bind to a detectable moiety. Exemplary detectable moieties include, but are not limited to, radioisotopes such as iodine 125, iodine 131, cesium 137, iridium 192 and cobalt 60, horseradish peroxidase, fluorescein isothiocyanate, biotin, alkaline phosphatase, chemiluminescent agents such as luminol, and the like. One skilled in the art may select a suitable detectable moiety to bind to the antibody or an antigen-binding portion thereof of the present application as desired to achieve different detection purposes.

In this specification and the claims, the terms “comprising,” and “comprises” and “comprise” mean “including, but not limited to” and are not intended to exclude other parts, additions, components or steps.

It is to be understood that the features, characteristics, components, or steps described in a particular aspect, embodiment, or example of the application are applicable to any other aspects, embodiments, or examples herein, unless indicated otherwise.

The present invention discloses antibodies that specifically bind to the mammal (human, primate, etc.) CLDN18.2, and provides use of such proteins in the treatment, screening, and detection, such as in the treatment of cancer. Those skilled in the art may appropriately improve the process parameter in view of the content herein. In particular, it should be noted that all similar alternatives and modifications will be apparent to those skilled in the art, and considered to be included within the scope of the present invention. Those skilled in the art will be able to achieve and apply the present invention by alterations or suitable variations and combinations of the methods and applications herein without departing from the context, spirit and scope of the invention.

The present invention will be described in further detail below in connection with the examples so as to make those skilled in the art better understand the technical solutions of the present invention.

### EXAMPLES

The following examples are used to illustrate the present application, but are not intended to limit the scope of the present application. Modifications or substitutions of the methods, steps or conditions of the present application are intended to fall within the scope of the present application without departing from the spirit and spirit thereof.

Unless otherwise specified, the chemical reagents used in the examples are all conventional commercially available reagents, and the technical means used in the examples are conventional means well known to those skilled in the art.

The examples do not encompass a detailed description of conventional methods, such as those used to construct vectors and plasmids, methods of inserting genes encoding proteins into vectors and plasmids, or methods of introducing plasmids into host cells. Such methods are well known to those of ordinary skill in the art and are described in many publications, see, for example, Sambrook, J., Fritsch, EF. and Maniais, T. (1989) Molecular Cloning: A Laboratory Manual, 2nd edition, Cold spring Harbor Laboratory Press.

### Example 1. Construction of Cell Lines Highly Expressing CLAUDIN 18.1 and 18.2 (CLDN18.1 and CLDN18.2)

The cell lines highly expressing the human CLDN18.1 and human CLDN18.2 were constructed by a stable cell line construction platform as follows:

Construction of plasmids pEnter-CLDN18.1-puromycin and pEnter-CLDN18.2-puromycin: Two small fragments of about 800 bp were amplified with primers using the CLDN18.1 cDNA (the encoded amino acid sequence of which is shown in SEQ ID NO: 13) and the CLDN18.2 cDNA (the encoded amino acid sequence of which is shown in SEQ ID NO: 14) as templates, respectively, and were subjected to gel recovery; The pENTER plasmid was digested with KpnI/HindIII and the large fragment of 7450 bp was recovered from the gel; The resultant two small fragments were respectively ligated into the recovered large fragment of the pENTER plasmid, which was digested with KpnI/HindIII, and the positive clones were sequenced. The plasmids having a correct sequence were designated as pEnter-CLDN18.1-puromycin and pEnter-CLDN18.2-puromycin, respectively.

The 293T cells (the cell bank of Xiehe) were seeded in two T25 culture flasks, each with a cell number of 2×10⁶. The next day, the 293T cell culture medium was replaced with 4 mL Opti-MEM (Thermofisher Scientific Cat31985070). The 5 μg of each of the plasmids pEnter-CLDN18.1-puromycin and pEnter-CLDN18.2-puromycin, each cloned with the human CLDN18.1 and CLDN18.2, were respectively added to Opti-MEM in a final volume of 500 μL. 7 μL of the transfection reagent PEI (3 μg/mL) was added to a new 500 μL Opti-MEM. Two Opti-MEM were mixed and allowed to stand at room temperature for 20 min before being added to the above cultured 4 ml 293T cells. On day 3, the cell culture medium was replaced with 5 mL DMEM high glucose medium. On day 4, 2 μg/mL puromycin was added for screening. After 2-3 days a large number of cells died. The fresh medium was added until the cells stably grew, and a single clone was screened, expanded and subjected to cryopreservation.

The cell lines stably expressing the gene of interest constructed in the present application were designated as 293T-18.1 cells and 293T-18.2 cells, respectively. The sequences of the proteins used were derived from published databases and the sequences of each protein were as follows:

SEQ ID NO: 13: Human CLDN18.1, NP_057453.1,
claudin-18 isotype 1 precursor [Homo sapiens]
MSTTTCQVVAFLLSILGLAGCIAATGMDMWSTQDLYDNPVTSVFQYEGLW
RSCVRQSSGFTECRPYFTILGLPAMLQAVRALMIVGIVLGAIGLLVSIFA
LKCIRIGSMEDSAKANMTLTSGIMFIVSGLCAIAGVSVFANMLVTNFWMS
TANMYTGMGGMVQTVQTRYTFGAALFVGWVAGGLTLIGGVMMCIACRGLA
PEETNYKAVSYHASGHSVAYKPGGFKASTGFGSNTKNKKIYDGGARTEDE
VQSYPSKHDYV
SEQ ID NO: 14: Human CLDN18.2, NP_001002026.1,
claudin-18 isoform 2 [Homo sapiens]
MSTTTCQVVAFLLSILGLAGCIAATGMDMWSTQDLYDNPVTSVFQYEGLW
RSCVRQSSGFTECRPYFTILGLPAMLQAVRALMIVGIVLGAIGLLVSIFA
LKCIRIGSMEDSAKANMTLTSGIMFIVSGLCAIAGVSVFANMLVTNFWMS
TANMYTGMGGMVQTVQTRYTFGAALFVGWVAGGLTLIGGVMMCIACRGLA
PEETNYKAVSYHASGHSVAYKPGGFKASTGFGSNTKNKKIYDGGARTEDE
VQSYPSKHDYV

### Example 2. Preparation of Hybridomas Producing Anti-CLDN18.2 Antibody

The preparation method was as follows:

Five BALB/C healthy female mice of seven to eight weeks old were selected for immunization. The 293T-18.2 cells were cultured, and washed three times with PBS to make sure that the serum was washed out. The cells were injected intraperitoneally to each mouse at a dose of 1×10⁷ cells/mouse in a total volume of 100-200 μl. Immunizations were performed every other week in the same amounts. Two weeks after the third immunization, the tail vein blood of the mice was centrifuged to obtain serum for titer determination (detected by flow cytometry). The optimal mice were selected when the titer met the fusion requirements.

Mice with high titer were selected for impulse immunization by intraperitoneal injection of the cells three days prior to fusion. On the day prior to the fusion, intraperitoneal macrophages of the normal Balb/c mice were taken as trophocytes.

The myeloma cells SP2/0 were thawed one week prior to the titer detection, and cultured in 5% CO₂ incubator at 37° C. The cells were passaged or the culture medium was replaced with fresh medium one day prior to fusion to maintain the best cell state.

The spleens from the immunized mice were fused with the myeloma cells SP2/0, and the resultant cells were added to a 96-well plate pre-plated with trophocytes. The fused cells were subjected to HAT selection (Galfre and Milstein, Methods Enzymol 1981; 73:3-46).

### Primary Screening of Anti-CLDN18.2 Hybridomas (ELISA)

The primary screening was performed by determining the titer of anti-CLDN18.2 antibodies secreted by the hybridomas using an enzyme-linked immunoassay (ELISA). Table 1 shows the test results for a part of the hybridoma supernatants.

TABLE 1

| Clone numberings of | Binding activity of |
|---|---|
| initial hybridomas | 293T-18.2 cells |
| 3C1 | 1.314 |
| 6B4 | 0.087 |
| 6E7 | 0.432 |
| 7B1 | 0.091 |
| 7D1 | 1.116 |

### Rescreening of Anti-CLDN18.2 Hybridomas (FACS)

The rescreening process was as follows:

The 293T-18.2 and 293T-18.1 cells were collected, washed once with the FACS buffer, and plated at 2-5×10⁵ cells per well into a 96-well deep-well plate. 50 μL hybridoma supernatant was added to each well and incubated for 2 hours. MAb362 (Kangyuan Bochuang Biotechnology (Beijing) Co., LTD) was selected as a positive control antibody.

Each well was washed twice with 400 μl FACS buffer. The secondary antibody of the PE anti-mouse IgG Fc (Biolegend, Cat405307) was added and incubated for 1 hour before detection by flow cytometry.

As shown in Table 2, five candidate monoclonal antibodies were obtained, and labeled as 6E7, 7D10, 7E12, 9A9, and 3C1, respectively.

TABLE 2
Rescreening Results of Anti-CLDN18.2 Hybridomas

| Clone numberings of | Binding activity of | Binding activity |
|---|---|---|
| initial hybridomas | 293T-18.2 cells | of 293T-18.1 cells |
| 3C1 | 3297 | 2451 |
| 6E7 | 1359 | 2673 |
| 7D10 | 2517 | 7.65 |
| 7E12 | 2620 | 1893 |
| 9A9 | 3267 | 3647 |
| Blank | 6.97 | 7.22 |
| Secondary antibody | 7.09 | 7.7 |
| Positive control | 1587 | 61.3 |

The human CLDN18.2 protein is highly homologous to the human CLDN18.1 protein, and it is a four transmembrane protein with a very short extracellular domain peptide sequence, very low immunogenicity, and very little possibility of producing specific antibodies, so that the probability of screening hybridomas capable of secreting antibodies recognizing CLDN18.2 is very low. A large portion of the antibodies in the supernatants of these hybridomas were those that bind both the human CLDN18.2 and the human CLDN18.1. In this example, a hybridoma cell 7D10 was obtained, and the antibody in the supernatant bound to the 293T-18.2 cells, but hardly bound to the 293T-18.1 cells.

### Antibody Screening and Purification of Hybridoma Cell 7D10 Subclones

The hybridoma cells 7D10 were plated in a 96-well plate and cultured in HT medium for 7-10 days. The positive clones were further screened by FACS. The positive cells were selected for expanded culture, and the supernatants were collected to perform antibody (the amino acid sequences of the heavy chain variable region and the light chain variable region are shown in SEQ ID NO:7 and SEQ ID NO:8, respectively) purification and concentration detection.

As shown in Table 3, a hybridoma monoclonal cell line 7D10-1 was obtained after further screening, and the antibody in the clone supernatant specifically bound to the 293T-18.2 cells and hardly bound to the 293T-18.1 cells. This means that the anti-CLDN18.2 antibody secreted by the hybridoma cells is a promising antibody.

TABLE 3
Screening of Hybridoma Cell 7D10 Subclones

| Clone numberings of | Binding activity of | Binding activity of |
|---|---|---|
| initial hybridomas | 293T-18.2 cells (FACS) | 293T-18.1 cells (FACS) |
| 7D10-1 | 1458 | 9.65 |
| 7D10-2 | 609 | 9.32 |
| 7D10-3 | 817 | 9.47 |
| 7D10-4 | 531 | 9.87 |
| 7D10-5 | 267 | 9.15 |

### Example 3. Binding Activity of Anti-CLDN18.2 Monoclonal Antibody Secreted by 7D10-1 Hybridoma to Human CLDN18 Positive Cells

Each of the T75 cell culture flasks respectively containing the 293T-18.1 and 293T-18.2 cells was washed once with FACS buffer, and resuspended in FACS buffer to adjust the cell concentration to 2×10⁶; The antibodies (including both the MAb362 and the anti-CLDN18.2 monoclonal antibody, with MAb362 as a control) were serially diluted 3.16 folds to 7 dilutions with 20 μg/ml as a starting concentration. 10 μL of each diluted antibody was added to 100 μL of each of the above cell samples (i.e., the 293T-18.1 and 293T-18.2 cells), mixed and incubated at room temperature for 1 hour.

Each well was washed twice with 400 μl FACS buffer. The secondary antibody of PE anti-mouse IgG Fc was added, and incubated for 1 hour before detection by flow cytometry. The results are shown in Table 4 and FIG. 1. The results of Table 4 show that both the anti-CLDN18.2 monoclonal antibody and the control antibody MAb362 specifically bind to the 293T-18.2 cells, and the EC50 of the anti-CLDN18.2 monoclonal antibody is much stronger than that of the control antibody MAb362, about three times that of the control antibody.

TABLE 4
Binding Activity of Anti-CLDN18.2 Monoclonal Antibody to
Human CLDN18 Positive Cells

| | Binding activity of 293T-18.2 cells | | Binding activity |
|---|---|---|---|
| | | MFI.Max (mean | of 293T-18.1 cells |
| Samples | EC 50 (μg/mL) | fluorescence intensity) | MFI.Max |
| MAb362 | 1.196 | 7126 | 8.29 |
| Anti-CLDN18.2 | 0.385 | 7993 | 15.1 |
| monoclonal | | | |
| antibody | | | |

### Example 4. Binding Activity of Anti-CLDN18.2 Chimeric Antibody to Human CLDN18 Positive Cells (FACS Detection)

### Preparation of Anti-CLDN18.2 Chimeric Antibody

The cDNA of 7D10-1 hybridoma cells were produced and sequenced by PCR amplification using the antibody primers in our laboratory. The nucleotide sequences encoding VH1 and VL1 of the murine anti-Claudin 18.2 antibody were finally obtained. The nucleic acid sequences encoding VH1 and VL1 of the anti-Claudin 18.2 antibody were constructed into the plasmids pUC57 Claudin 18.2 VH1 and pUC57 Claudin 18.2 VL1 (General Biological Systems (Anhui) Co., Ltd.) by the full synthesis method. The anti-CLDN18.2 VH1 and the anti-CLDN18.2 VL1 fragments were amplified by the Gold Mix PCR kit (TSINGKE Co., Ltd.) according to the instructions of the kit using the pUC57 Claudin 18.2 VH1 and pUC57 Claudin 18.2 VL1 as templates, respectively. The amplified products were about 0.4 kb in size. The vector plasmids pQKX1 and pQKX2 were digested with the restriction endonuclease SapI (NEB, R0569S). The resulting PCR amplification products and the digested vectors were recombinantly ligated to obtain the heavy chain expression vector pQK CLDN18.2 H1 and the light chain expression vector pQK CLDN18.2 L1 using a BM seamless cloning kit (Bordeaux) according to the instructions of the kit.

The 293Fv cells (Cell Resource Center, Institute of Basic Medicine, Chinese Academy of Medical Sciences) were diluted to 1.5×10⁶ cells/mL in a final volume of 200 ml, and cultured in a shaker at 37° C. for 24 hours. The plasmids pQK CLDN18.2 H1 and pQK CLDN18.2 L1 were diluted with a 10% transfection volume (i.e., the volume of the transfected cell) of the fresh medium to a concentration of 1 μg/mL. 200 μl ( 1/1000 of cell volume) of PEI at 3 mg/mL was added to the diluted plasmid, immediately vortexed for 10 seconds, and left at room temperature for 15 minutes. The plasmid/PEI mixture was added dropwise to the cell culture medium (the culture flask was gently shaken while adding dropwise), and placed into a shaker for culture. After 7 days, the culture supernatant was collected and through a 0.4 um filter membrane, and the antibody was captured from the culture supernatant using a Mab sure Lx 5 ml purification column with the flow rate of 3 ml/min. The column was equilibrated with 5 column volumes of the equilibrium liquid 20 mM PB+150 mM NaCl, pH7.4, and loaded. The column was rinsed with 20 mM PB+150 mM NaCl, pH7.4 when the loading was ended, and eluted by using the eluent of 50 mM citric acid pH3.0. The eluted antibody was neutralized with 1M Tris-HCl, pH9.0, and collected (the amino acid sequences of the heavy chain and the light chain are shown in SEQ ID NO:9 and SEQ ID NO:10, respectively).

The 293T-18.2 cells in a T75 cell culture flask were collected, washed once with FACS buffer, and resuspended in FACS buffer to adjust the cell concentration to 2×10⁶; The antibodies (including both the MAb362 and the anti-CLDN18.2 chimeric antibody, with MAb362 as a control) were serially diluted 3.16 folds to 7 dilutions with 20 μg/ml as a starting concentration. 10 μL of each diluted antibody was added to 100 μL of the above cell samples, mixed and incubated at room temperature for 1 hour.

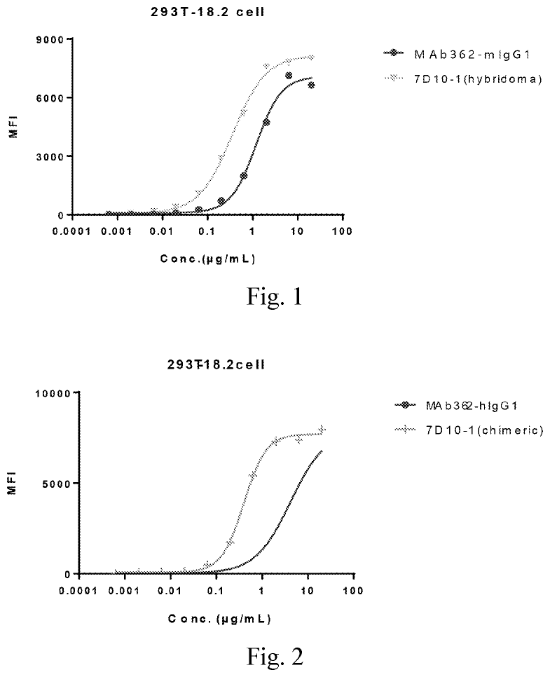

Each well was washed twice with 400 μl FACS buffer. The secondary antibody of the PE anti-human IgG Fc (Biolegend, Cat409304) was added and incubated for 1 hour before detection by flow cytometry. The results are shown in Table 5 and FIG. 2. The results in Table 5 show that both the anti-CLDN18.2 chimeric antibody and the control antibody MAb362 specifically bind to the 293T-18.2 cells, and the EC50 of the anti-CLDN18.2 chimeric antibody is much stronger than that of the control antibody MAb362, about ten times that of the control antibody.

TABLE 5
Binding Activity of Anti-CLDN18.2 Chimeric Antibody to
Human CLDN18.2 Positive Cells

| | Binding activity of 293T-18.2 cells (FACS) | |
|---|---|---|
| Samples | EC 50 (μg/mL) | MFI.Max |
| MAb362 | 4.16 | 6753 |
| 7D10-1 chimeric antibody | 0.3996 | 7954 |

### Example 5. Binding Activity of Anti-CLDN18.2 Humanized Antibodies to Human CLDN18.2 Positive Cells (FACS Detection)

### Preparation of Anti-CLDN18.2 Humanized Antibody

The nucleotide sequences encoding VH2 and VL2 of the anti-Claudin 18.2 humanized antibody were designed by the inventors of the present application. The nucleotide sequences encoding VH2 and VL2 of the anti-Claudin 18.2 humanized antibody were constructed into the plasmids pUC57 Claudin 18.2 VH2 and pUC57 Claudin 18.2 VL2 (General Biological Systems (Anhui) Co., Ltd.) by the full synthesis method. The anti-CLDN18.2 VH2 and the anti-CLDN18.2 VL2 fragments were amplified by the Gold Mix PCR kit (TSINGKE Co., Ltd.) according to the instructions of the kit using the pUC57 Claudin 18.2 VH2 and pUC57 Claudin 18.2 VL2 as templates, respectively. The amplified products were about 0.4 kb in size. The vector plasmids pQKX1 and pQKX2 were digested with the restriction endonuclease SapI (NEB, R0569S). The resulting PCR amplification products and the digested vectors were recombinantly ligated to obtain the heavy chain expression vector pQK CLDN18.2 H2 and the light chain expression vector pQK CLDN18.2 L2 using a BM seamless cloning kit (Bordeaux) according to the instructions of the kit.

The 293Fv cells were diluted to 1.5×10⁶ cells/mL in a final volume of 200 ml, and cultured in a shaker at 37° C. for 24 hours. The plasmids pQK CLDN18.2 H2 and pQK CLDN18.2 L2 were diluted with a 10% transfection volume (i.e., the volume of the transfected cell) of the fresh medium to a concentration of 1 μg/mL. 200 μl ( 1/1000 of cell volume) of PEI at 3 mg/mL was added to the diluted plasmid, immediately vortexed for 10 seconds, and left at room temperature for 15 minutes. The plasmid/PEI mixture was added dropwise to the cell culture medium (the culture flask was gently shaken while adding dropwise), and placed into a shaker for culture. After 7 days, the culture supernatant was collected and through a 0.4 μm filter membrane, and the antibody was captured from the culture supernatant using a Mab sure Lx 5 ml purification column with the flow rate of 3 ml/min. The column was equilibrated with 5 column volumes of the equilibrium liquid 20 mM PB+150 mM NaCl, pH7.4, and loaded. The column was rinsed with 20 mM PB+150 mM NaCl, pH7.4 when the loading was ended, and eluted by using the eluent of 50 mM citric acid pH3.0. The eluted antibody was neutralized with 1M Tris-HCl, pH9.0, and collected (the amino acid sequences of the heavy chain variable region and the light chain variable region are shown in SEQ ID NO:11 and SEQ ID NO:12, respectively).

The 293T-18.2 cells in a T75 cell culture flask were collected, washed once with FACS buffer, and resuspended in FACS buffer to adjust the cell concentration to 2×10⁶; The antibodies (including both the MAb362 and the anti-CLDN18.2 humanized antibody, with MAb362 as a control) were serially diluted 3.16 folds to 7 dilutions with 20 μg/ml as a starting concentration. 10 μL of each diluted antibody was added to 100 μL of the above cell samples, mixed and incubated at room temperature for 1 hour.

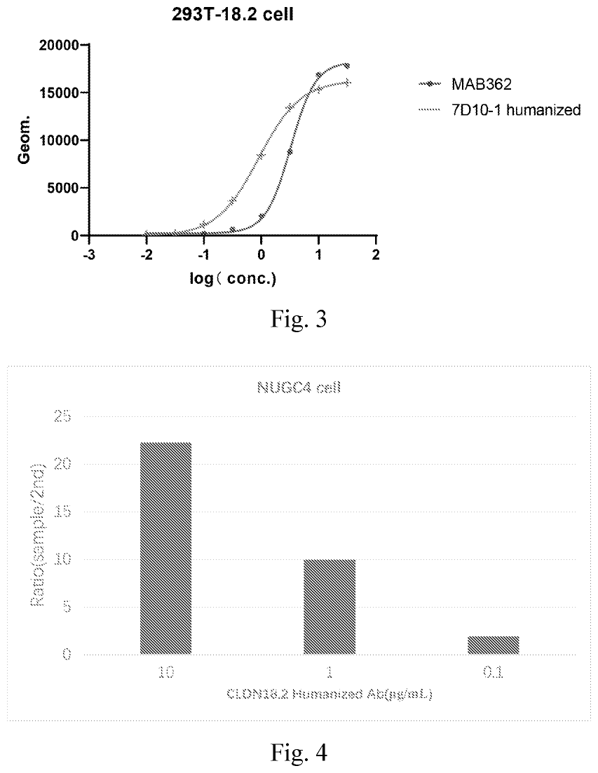

Each well was washed twice with 400 μl FACS buffer. The secondary antibody of the PE anti-human IgG Fc was added and incubated for 1 hour before detection by flow cytometry. The results are shown in Table 6 and FIG. 3. The results in Table 6 show that both the anti-CLDN18.2 humanized antibody and the control antibody MAb362 specifically bind to the 293T-18.2 cells, and the EC50 of the anti-CLDN18.2 humanized antibody is much stronger than that of the control antibody MAb362, about four times that of the control antibody.

TABLE 6
Binding Activity of Anti-CLDN18.2 Humanized Antibody
to Human CLDN18.2 Positive Cells

| | Binding activity of 293T-18.2 cells (FACS) | |
|---|---|---|
| Samples | EC 50 (μg/mL) | MFI.Max |
| MAb362 | 3.259 | 17793 |
| 7D10-1 humanized antibody | 0.9092 | 16034 |

### Example 6. Activity of Anti-CLDN18.2 Humanized Antibody

### Binding Activity of Anti-CLDN18.2 Humanized Antibody to Human Gastric Cancer Cell Line NUGC4

NUGC4 cells in a T75 cell culture flask (China American Coronaceae Biotechnology Beijing Co., Ltd.) were collected, washed once with FACS buffer, and resuspended in FACS buffer to adjust the cell concentration to 2×10⁶; The anti-CLDN18.2 humanized antibody was serially diluted 10 folds to 3 dilutions with 20 μg/ml as a starting concentration. 10 μL of each diluted antibody was added to 100 μL of the above NUGC4 cell samples, mixed and incubated at room temperature for 1 hour.

Each well was washed twice with 400 μl FACS buffer. The secondary antibody of the PE anti-human IgG Fc was added and incubated for 1 hour before detection by flow cytometry. The results are shown in FIG. 4. FIG. 4 shows specific binding of the anti-CLDN18.2 humanized antibody to the NUGC4 cells.

### ADCC Activity of Anti-CLDN18.2 Humanized Antibody on Human Gastric Cancer Cell Line NUGC4

A bottle (T75) of NUGC4 cells was collected and adjusted to a density of 2×10⁵ cells/ml. 50 μl of DMEM containing 10% low IgG FBS was added to each well of a real-time cell analyzer (RTCA) plate, and the plate was assayed by a real-time cell analyzer and the baseline was determined. The culture medium in wells was aspirated, and NUGC4 single cell suspension was added at 100 μl per well. After settled to the bottom of the plate, the cells were placed in the RTCA instrument (in a cell incubator), incubated overnight and examined for cell growth.

Human peripheral blood was collected, diluted with PBS at a rate of 1:1 and plated in equal volumes on 4 ml Ficoll-Pague™ plus (at a density of 1.077±0.001 g/ml, GE, 17-1440-03) in a 15 ml centrifuge tube. After centrifugation at 800 g for 20 min at room temperature, the buffy coat layer was aspirated to obtain PBMCs. The resultant PBMCs were washed twice with PBS and then adjusted to a cell density of 4×10⁶ cells/ml.

Both the anti-CLDN18.2 humanized antibody and the human IgG1 isotype control antibody (Biolegend, Cat403502) were serially diluted 2.57 folds with PBS to 6 dilutions with 580 μg/ml (4 μM) as a starting concentration.

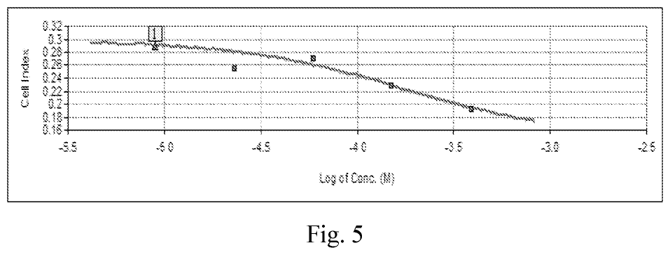

The RTCA instrument was suspended, and the RTCA plate was taken out. 50 μl of each of the different antibodies at different dilutions and 50 μl PBMC were added in duplicate, and both target cell control (Blank) and antibody-free control (PBMC) were set. The RTCA plate was left until the cells were settled to the bottom of the plate, and was subjected to continuous detection. EC50 of ADCC of the anti-CLDN18.2 humanized antibody against the human gastric cancer cell NUGC4 was calculated to be 0.18 mM (see FIG. 5), while the IgG1 isotype control antibody did not produce an ADCC effect against NUGC4, resulting in a concentration-independent gradient trend.

It is to be understood that while the inventions referred to have been described in the foregoing specific embodiments, these inventions are not limited to the specific content of these specific embodiments. It will be apparent to those skilled in the art that various equivalent variations may be made to the technical features contained in the inventions referred to therein without departing from the spirit of the inventions herein, and these variations are intended to be within the scope of the inventions described as.

What is claimed is:

1. An antibody or an antigen-binding portion thereof that specifically binds to CLDN18.2, comprising a heavy chain variable region comprising any one or more of the HCDR1, HCDR2, and HCDR3 sequences, wherein the HCDR1 sequence is GFSFSSFG (SEQ ID NO:1), the HCDR2 sequence is ISSGSRTI (SEQ ID NO:2), and the HCDR3 sequence is TRYYYGNSFDY (SEQ ID NO:3).

2. The antibody or the antigen-binding portion thereof of claim 1, further comprising a light chain variable region comprising any one or more of the LCDR1, LCDR2, and LCDR3 sequences, wherein the LCDR1 sequence is QSLFNTGNQKNY (SEQ ID NO: 4), the LCDR2 sequence is WAS (SEQ ID NO: 5), and the LCDR3 sequence is QNNYNFPLT (SEQ ID NO: 6).

3. The antibody or the antigen-binding portion thereof of claim 1 or 2, wherein the antibody is a murine antibody, optionally, the amino acid sequence of the heavy chain variable region is as shown in SEQ ID NO:7, and/or the amino acid sequence of the light chain variable region is as shown in SEQ ID NO:8.

4. The antibody or the antigen-binding portion thereof of claim 1 or 2, wherein the CLDN18.2 is a primate CLDN18.2.

5. A pharmaceutical composition comprising the antibody or the antigen-binding portion thereof of claim 1 and a pharmaceutically acceptable carrier, optionally, the pharmaceutical composition further comprises one or more other active ingredients, such as an agent for the treatment of tumors.

6. A nucleic acid molecule encoding the antibody or the antigen-binding portion thereof of claim 1.

7. An expression vector comprising the nucleic acid molecule of claim 6.

8. A host cell comprising the expression vector of claim 7.

9. (canceled)

10. A detection reagent or a kit comprising the antibody or the antigen-binding portion thereof of claim 1.

11. A method of preventing and/or treating a CLDN18.2-related disease comprising administering to a subject in need thereof a therapeutically effective amount of the antibody or the antigen-binding portion thereof of claim 1, optionally the CLDN18.2-related disease is a tumor, optionally the tumor is selected from one or more of: gastric cancer, pancreatic cancer, intestinal cancer, esophageal cancer, liver cancer, ovarian cancer, lung cancer and bladder cancer, and metastatic tumors thereof.

12. (canceled)

13. A method of preventing and/or treating a CLDN18.2-related disease comprising administering to a subject in need thereof a therapeutically effective amount of the pharmaceutical composition of claim 5.

14. The antibody or the antigen-binding portion thereof of claim 1, the antigen-binding portion is selected from the group consisting of a Fab fragment, a Fab′ fragment, a F(ab′)2 fragment, a scFv fragment, a Fd fragment, or a single domain antibody.

15. The antibody or the antigen-binding portion thereof of claim 1, wherein the antibody is a chimeric antibody, optionally, the chimeric antibody comprises a heavy chain having an amino acid sequence as shown in SEQ ID NO:9, and/or a light chain having an amino acid sequence as shown in SEQ ID NO:10.

16. The antibody or the antigen-binding portion thereof of claim 1, wherein the antibody is a humanized antibody, optionally, the amino acid sequence of the heavy chain variable region is as shown in SEQ ID NO:11, and/or the amino acid sequence of the light chain variable region is as shown in SEQ ID NO:12.

17. A pharmaceutical composition comprising the antibody or the antigen-binding portion thereof of claim 2 and a pharmaceutically acceptable carrier,
wherein the pharmaceutical composition optionally further comprises one or more other active ingredients.

18. A nucleic acid molecule encoding the antibody or the antigen-binding portion thereof of claim 2.

19. A host cell comprising the nucleic acid molecule of claim 6.

20. A detection reagent or a kit comprising the antibody or the antigen-binding portion thereof of claim 2.

21. A method of preventing and/or treating a CLDN18.2-related disease comprising administering to a subject in need thereof a therapeutically effective amount of the expression vector of claim 7.

22. The antibody or the antigen-binding portion thereof of claim 4, wherein the primate CLDN18.2 is selected from the group consisting of a human CLDN18.2 or a monkey CLDN18.2.

23. The method of claim 13, wherein the CLDN18.2-related disease is a tumor selected from one or more of: gastric cancer, pancreatic cancer, intestinal cancer, esophageal cancer, liver cancer, ovarian cancer, lung cancer and bladder cancer, and metastatic tumors thereof.

24. The method of claim 21, wherein the CLDN18.2-related disease is a tumor selected from one or more of: gastric cancer, pancreatic cancer, intestinal cancer, esophageal cancer, liver cancer, ovarian cancer, lung cancer and bladder cancer, and metastatic tumors thereof.
